(19) United States
(12) Patent Application Publication
(10) Pub. No.: US 2005/0196368 A1
(43) Pub. Date: Sep. 8, 2005

(54) Cosmetic composition comprising at least one anionic fixing polymer and at least one nonionic compound, and a process for shaping keratin fibers therewith by applying heat

(76) Inventors: Ludivine Laurent, Courbevoie (FR); Laurent Cochonneau, Laigne-en-Belin (FR); Cecile Bebot, Clichy (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/056,113

(22) Filed: Feb. 14, 2005

Related U.S. Application Data
(60) Provisional application No. 60/559,027, filed on Apr. 5, 2004.

(30) Foreign Application Priority Data
Feb. 12, 2004 (FR) 04 01401

(51) Int. Cl. A61K 7/06; A61K 7/11; A61K 7/09

(52) U.S. Cl. 424/70.2; 424/70.12

(57) ABSTRACT

The present disclosure relates to a process for shaping keratin fibers, for instance, human keratin fibers such as the hair, comprising applying to the keratin fibers a cosmetic composition comprising at least one anionic fixing polymer and at least one nonionic compound chosen from fatty alcohols, polyols and silicones, and after an optional action time, placing the keratin fibers in contact with heat that shapes the keratin fibers; the present disclosure also relates to the composition used in the process.

This application claims benefit of U.S. Provisional Application No. 60/559,027, filed Apr. 5, 2004, and of French Patent Application No. 04/01401, the contents of both of which are incorporated herein by reference.

The present disclosure relates to a process for shaping keratin fibers, for instance human keratin fibers such as the hair, comprising the application to the keratin fibers of a composition comprising at least one anionic fixing polymer and at least one nonionic compound; and the sequential or simultaneous application of heat to shape keratin fibers.

The use of heat to shape keratin fibers, such as in the form of smoothing irons, curling irons or embossing irons, is known. It is also known to shape keratin fibers using heat, e.g., using devices such as listed above, in combination with compositions for instance, comprising polymers, optionally anionic polymers, as described in French Patent Application No. FR 03/05636. The results obtained, however, may be accompanied by a formation on the hair of unattractive residues in the form of whitish particles.

Accordingly, it would be desirable to find a process for shaping keratin fibers that does not generate such unattractive residues. Advantageously and unexpectedly, the present inventors have discovered that this problem of the prior art can be avoided by performing a process that comprises applying to keratin fibers a composition comprising at least one anionic fixing polymer and at least one nonionic compound, and placing the keratin fibers; sequentially or simultaneously, in contact with heat to shape the keratin fibers.

The process according to the present disclosure can be easy to perform: it can allow easy shaping of the desired hairstyle, and can lead to a good level of fixing of the keratin fibers, which holds throughout the day or even for several days, and the effect obtained can also show good resistance to moisture and be easy to remove by shampooing; furthermore, this process can make it possible to avoid the formation on the keratin fibers of unattractive whitish particles. This process can also make it possible to give the hair good cosmetic properties such as softness, easy disentangling and sheen.

Other characteristics, aspects, subjects and benefits of the present disclosure, will emerge more clearly upon reading the description and the examples that follow.

One aspect of the present disclosure is thus a process for shaping keratin fibers.

The process according to the present disclosure is a process for shaping keratin fibers, for instance, human keratin fibers, such as the hair, comprising:
applying to the keratin fibers a cosmetic composition comprising at least one anionic fixing polymer and at least one nonionic compound chosen from fatty alcohols, polyols and silicones, and
after an optional action time, placing the keratin fibers in contact with heat to shape the keratin fibers.

Another aspect of the present disclosure is a cosmetic composition comprising at least one anionic fixing polymer and at least one nonionic compound chosen from fatty alcohols, polyols and silicones, wherein the weight ratio of the at least one anionic fixing polymer to the at least one nonionic compound is greater than or equal to 1.

As used herein, the term “process for shaping keratin materials” is understood to mean any process for modifying the initial appearance of the hairstyle: it may thus be a process for curling or embossing the hair, or a process for smoothing or straightening the hair.

The at least one anionic fixing polymer that may be used in the compositions of the present disclosure includes, for example, branched block copolymers comprising, as main monomers, at least one C₁₋₂₀ alkyl acrylate and/or at least one N-mono- or N,N-di(C₂₋₁₂alkyl)(meth)acrylamide, and acrylic acid and/or methacrylic acid.

Another aspect of the present disclosure is the use of at least one nonionic compound chosen from fatty alcohols, polyols and silicones to reduce the unattractive residues of anionic fixing polymers during the placing of these polymers in contact with heat.

When the cosmetic composition as disclosed herein is applied to the keratin fibers and before applying heat, a period of “action time” ranging from 30 seconds to 10 minutes may be observed.

Placing the keratin fibers in contact with heat can be performed using heat in any form that can shape the keratin fibers. For example, hair styling appliances can make it possible to heat the keratin fibers to a temperature ranging from 50° C. to 250° C., such as from 100° C. to 230° C. For instance, heat can be applied to the hair in order to curl or emboss the hair, or alternatively to smooth or straighten the hair, and can be in the form of a heating device, such as smoothing irons, curling irons or crimping irons.

The application of the heat may take place by successive touches or by sliding the appliance along the fibers.

According to one embodiment of the present disclosure, the heat can be in the form of an iron that has a reservoir allowing the direct delivery of the inventive composition onto the fibers.

The cosmetic composition to be applied to the hair comprises at least one anionic fixing polymer and at least one nonionic compound chosen from fatty alcohols, silicones and polyols. In one embodiment of the present disclosure, the weight ratio of the at least one anionic fixing polymer to the at least one nonionic compound is greater than or equal to 1.

As used herein, the term “fixing polymer” is understood to mean any polymer capable of giving the hair a shape or of modifying its original shape.

Among the anionic fixing polymers that may be used in the cosmetic composition as disclosed herein, non-limiting mention may be made of polymers comprising groups derived from carboxylic, sulfonic or phosphoric acid and that have a number-average molecular mass ranging from 500 to 5,000,000.

The carboxylic groups are provided by unsaturated monocarboxylic or dicarboxylic acid monomers, such as those of formula (I): 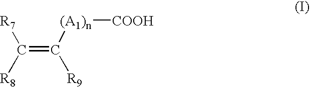
wherein
n is an integer from 0 to 10,
A₁ is a methylene group, optionally connected to the carbon atom of the unsaturated group, or to the neighboring methylene group when n is greater than 1, via a heteroatom, such as oxygen or sulfur,
R₇ is chosen from a hydrogen atom and phenyl and benzyl groups,
R₈ is chosen from a hydrogen atom and lower alkyl and carboxyl groups,
R₉ is chosen from a hydrogen atom, lower alkyl groups, —CH₂—COOH groups, phenyl groups and benzyl groups.

In formula (I), a lower alkyl group can mean, for example, a group comprising 1 to 4 carbon atoms, such as methyl and ethyl.

Among the anionic fixing polymers comprising carboxylic groups that may be used according to the present disclosure, non-limiting mention may be made of:

A) acrylic or methacrylic acid homo- or copolymers, or salts thereof, for example, the products sold under the names Versicol® E or K by the company Allied Colloid and Ultrahold® by the company BASF, the copolymers of acrylic acid and of acrylamide sold in the form of their sodium salts under the names Reten 421, 423 or 425 by the company Hercules, the sodium salts of polyhydroxycarboxylic acids.

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No. 1 222 944, and German Patent Application No. 2 330 956, the copolymers of this type comprising an optionally N-alkylated and/or hydroxyalkylated acrylamide unit in their chain as described, for instance, in Luxembourg Patent Application Nos. 75370 and 75371, or sold under the name Quadramer by the company American Cyanamid. Non-limiting mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF. Further non-limiting mention may also be made of methacrylic acid/acrylic acid/ethyl acrylate/methyl methacrylate copolymers as an aqueous dispersion, sold under the name Amerhold® DR 25 by the company Amerchol.

C) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those comprising at least 5 carbon atoms, it being possible for these polymers optionally to be grafted or crosslinked, or alternatively another vinyl, allylic or methallylic ester monomer of an α- or β-cyclic carboxylic acid. Such polymers are described, inter alia, in French Patent Nos. 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) copolymers derived from C₄-C₈ monounsaturated carboxylic acids or anhydrides chosen from:
copolymers comprising (i) at least one acid chosen from maleic, fumaric and itaconic acids and anhydrides and (ii) at least one monomer chosen from vinyl esters, vinyl ethers, vinyl halides, phenylvinyl derivatives, acrylic acid and its esters, the anhydride functional groups of these copolymers optionally being monoesterified or monoamidated. Such polymers are described, for instance, in U.S. Pat. Nos. 2,047,398, 2,723,248, and 2,102,113 and British Patent No. GB 839 805. Commercial products include, for example, those sold under the names Gantrez® AN or ES by the company ISP,
copolymers comprising (i) at least one unit chosen from maleic, citraconic and itaconic anhydride units and (ii) at least one monomer chosen from allylic or methallylic esters optionally comprising at least one entity chosen from acrylamide, methacrylamide, α-olefin, acrylic and methacrylic ester groups, acrylic and methacrylic acid groups and vinylpyrrolidone groups in their chain, the anhydride functional groups of these copolymers optionally being monoesterified or monoamidated. These polymers are described, for example, in French Patent Nos. FR 2 350 384 and FR 2 357 241.

E) polyacrylamides comprising carboxylate groups.

The homopolymers and copolymers comprising sulfonic groups are polymers comprising vinylsulfonic, styrenesulfonic, naphthalenesulfonic or acrylamidoalkylsulfonic units. Among these polymers that can be used as disclosed herein, non-limiting mention may be made of:
polyvinylsulfonic acid salts having a molecular weight ranging from 1,000 to 100,000, as well as the copolymers with an unsaturated comonomer such as acrylic or methacrylic acids and their esters, as well as acrylamide or its derivatives, vinyl ethers and vinylpyrrolidone;
polystyrenesulfonic acid salts such as the sodium salts that are sold under the names Flexan® 500 and Flexan® 130 by National Starch. These compounds are described in French Patent No. FR 2 198 719;
polyacrylamidesulfonic acid salts, such as those mentioned in U.S. Pat. No. 4,128,631, for example, polyacrylamidoethylpropanesulfonic acid sold under the name Cosmedia Polymer HSP 1180 by Henkel.

As another anionic fixing polymer that can be used according to the present disclosure, non-limiting mention may be made of the branched block anionic polymer sold under the name Fixate G100 by the company Noveon.

The anionic fixing polymers may also be chosen from, for example, acrylic acid copolymers, such as the acrylic acid/ethyl acrylate/N-tert-butylacrylamide terpolymers sold, for instance, under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold, for instance, under the name. Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold under the name Gantrez® ES 425 by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex A® by the company BASF, and the polymer sold under the name Fixate G100 by the company Noveon.

In some embodiments, the anionic fixing polymers that may be used in the context of the present disclosure may be chosen from the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone LM by the company ISP, and the polymer sold under the name Fixate G100 by the company Noveon.

According to the present disclosure, it is also possible to use anionic fixing polymers of grafted silicone type comprising a polysiloxane portion and a portion comprising a nonsilicone organic chain, one of the two portions constituting the main chain of the polymer, and the other being grafted onto the said main chain. These polymers are described, for example, in European Patent Application Nos. EP-A-0 412 704, EP-A-0 412 707, EP-A-0 640 105 and EP-A-0 582 152, International Patent Application Nos. WO 95/00578, and WO 93/23009, and U.S. Pat. Nos. 4,693,935, 4,728,571 and 4,972,037.

Such polymers are, for example, copolymers that can be obtained by free radical polymerization from the monomer mixture formed from:
a) from 50% to 90% by weight of tert-butyl acrylate;
b) from 0% to 40% by weight of acrylic acid;
c) from 5% to 40% by weight of a silicone macromer of formula: 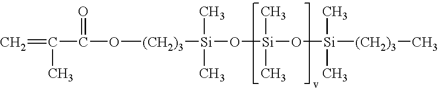
   wherein v is a number ranging from 5 to 700; the weight percentages being calculated relative to the total weight of the monomers.

Other examples of grafted silicone polymers include, for example, polydimethylsiloxanes (PDMSs) onto which are grafted, via a thiopropylene-type connecting chain, mixed polymer units of the poly((meth)acrylic acid) type and of the poly(alkyl (meth)acrylate) type.

Another type of anionic silicone fixing polymer that may be used is, for example, the product Luviflex® Silk, sold by the company BASF.

Functionalized or non-functionalized, silicone or non-silicone, anionic polyurethanes may also be used as fixing polymers. Among the polyurethanes that may be used, non-limiting mention may be made of those disclosed in European Patent Nos. EP 0 751 162, EP 0 637 600, EP 0 648 485, EP 0 656 021, and EP 0 619 111, French Patent No. FR 2 743 297, and International Patent No. WO 94/03510.

Among polyurethanes that that may be used according to the present disclosure, further non-limiting mention may be made of the products sold under the names Luviset Pur® and Luviset® Si-Pur by the company BASF.

Among the anionic fixing polymers that may be used for the purposes of the present disclosure are also copolymers comprising branched blocks comprising, as main monomers, at least one C₁₋₂₀ alkyl acrylate and/or at least one N-mono- or N,N-di(C₂₋₁₂ alkyl)(meth)acrylamide, and acrylic acid and/or methacrylic acid. These branched block copolymers have a structure comprising hydrophobic blocks to which are attached, for instance via difunctional units, a certain number of more hydrophilic blocks. These copolymers have at least two glass transition temperatures. These copolymers comprise:
from 26% to 36 mol % of acrylic acid
from 27.5% to 30.5 mol % of n-butyl acrylate
from 33.3% to 45.3 mol % of methacrylic acid
from 0.48% to 0.92 mol % of allyl methacrylate.

The most hydrophobic blocks have a molecular weight ranging from 10,000 to 100,000 and the most hydrophilic blocks have a molecular weight ranging from 1,000 to 100,000 daltons.

The above discussed fixing film-forming polymers are in anionic form, i.e., they are converted into salts by partial or total neutralization of the (meth)acrylic acid groups. Non-limiting examples of neutralizers that may be mentioned include 2-amino-2-methyl-1-propanol and sodium hydroxide. They are described, for example, in International Patent Application No. WO 00/40628 and sold, for example, under the names EX-SDR-26® and EX-SDR-45® by the company Goodrich.

The at least one anionic fixing polymer is can be present in the compositions according to the present disclosure in an amount ranging from 0.1% to 20%, such as from 0.5% to 10%, for instance from 0.6% to 5% by weight, relative to the total weight of the composition.

In one aspect of the present disclosure, the at least one anionic fixing polymer can be chosen from branched block copolymers comprising, as main monomers, at least one C₁₋₂₀ alkyl acrylate and/or at least one N-mono- or N,N-di(C₂₋₁₂ alkyl)(meth)acrylamide, and acrylic acid and/or methacrylic acid, and can be present in an amount, for example, ranging from 0.6% to 5% by weight, relative to the total weight of the composition.

Nonionic compounds that may be used in the cosmetic compositions as disclosed herein include optionally ethoxylated fatty alcohols, polyols and silicones.

As used herein, the term “fatty alcohols” is understood to mean linear or branched, such as linear, optionally unsaturated alcohols comprising from 4 to 30, for instance from 8 to 22, such as from 12 to 22 carbon atoms.

These alcohols may be ethoxylated, in which case they comprise from 2 to 10 EO groups. For example, the ethoxylated fatty alcohols that may be used according to the present disclosure have an HLB>5; these fatty alcohols may be used, for instance, in the compositions according to the present disclosure.

The polyols that may be used in the context of the present disclosure can be, for instance C₃-C₁₀ polyols, comprising from 2 to 5 OH groups. For example, in one embodiment of the present disclosure, they may be chosen from glycerol, glycol, propylene glycol, dipropylene glycol, butylene glycol and butyl diglycol groups.

The silicones that may be used in the context of the present disclosure may be linear, cyclic, branched or unbranched, and volatile or non-volatile. They may be in the form of oils, resins or gums, such as, for instance, polyorganosiloxanes. The organopolysiloxanes are defined in greater detail in Walter Noll's “Chemistry and Technology of Silicones” (1968) Academic Press. They can be volatile or non-volatile.

When they are volatile, the silicones can be chosen from those having a boiling point ranging from 60° C. to 260° C.; for instance, the silicones can be chosen from:
(i) cyclic silicones comprising from 3 to 7, such as from 4 to 5 silicon atoms. These are, for example, octamethylcyclotetrasiloxane sold for instance under the name Volatile Silicone 7207 by Union Carbide or Silbione 70045 V 2 by Rhodia Chimie, decamethylcyclopentasiloxane sold under the name Volatile Silicone 7158 by Union Carbide, and Silbione 70045 V 5 by Rhodia, and mixtures thereof. Non-limiting mention may also be made of cyclocopolymers of the dimethylsiloxane/methylalkylsiloxane type, such as Volatile Silicone FZ 3109 sold by the company Union Carbide, having the chemical structure:

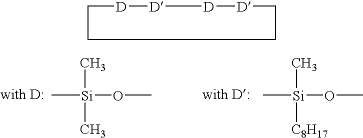

Non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones comprising from 2 to 9 silicon atoms and having a viscosity of less than or equal to 5×10⁻⁶ m²/s at 25° C. An example is decamethyltetrasiloxane sold, for instance, under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics.”

Among the non-volatile silicones that may be used as disclosed herein, non-limiting mention may be made of polyalkylsiloxanes, polyarylsiloxanes, polyalkylarylsiloxanes, silicone gums and resins, polyorganosiloxanes modified with organofunctional groups, and mixtures thereof.

The organomodified silicones that may be used in accordance with the present disclosure are silicones as defined above and comprising in their structure at least one organofunctional group attached via a hydrocarbon-based group. Among the organomodified silicones that may be used, non-limiting mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200. These silicones are used, for example, in at least one embodiment of the present disclosure;
thiol groups such as the products sold under the names “GP 72 A” and “GP 71” from Genesee;
alkoxylated groups such as the product sold under the name “Silicone Copolymer F-755” by SWS Silicones and Abil Wax® 2428, 2434 and 2440 by the company Goldschmidt;
hydroxylated groups such as the polyorganosiloxanes comprising a hydroxyalkyl functional group, described in French Patent Application No. FR-A-85/16334;
acyloxyalkyl groups such as, for example, the polyorganosiloxanes described in U.S. Pat. No. 4,957,732;
hydroxyacylamino groups, such as the polyorganosiloxanes described in European Patent Application No. EP 342 834. Further non-limiting mention may be made, for example, of the product Q2-8413 from the company Dow Corning.

The silicone oils that may be used in the compositions according to the present disclosure are volatile or non-volatile polymethylsiloxanes comprising a linear or cyclic silicone chain, which are liquid or pasty at room temperature, such as, for instance, cyclopolydimethylsiloxanes (cyclomethicones) such as cyclohexasiloxane; polydimethylsiloxanes comprising alkyl, alkoxy or phenyl groups, which are pendent or at the end of a silicone chain, these groups comprising from 2 to 24 carbon atoms; phenylsilicones, for instance phenyl trimethicones, phenyl dimethicones, phenyltrimethylsiloxydiphenylsiloxanes, diphenyl dimethicones, diphenylmethyldiphenyltrisiloxanes, polymethylphenylsiloxanes; and mixtures thereof.

The silicone gums that may be used in the compositions according to the present disclosure are polydiorganosiloxanes with a high molecular mass, ranging from 200,000 to 2,000,000, used alone or as a mixture in a solvent chosen from volatile silicones, polydimethylsiloxane oils, polyphenylmethylsiloxane oils, polydiphenyldimethylsiloxane oils, isoparaffins, methylene chloride, pentane and hydrocarbons, or mixtures thereof.

In the case of gums, for example, a silicone gum with a molecular weight of less than 1,500,000 may be used. The silicone gums can be, for example, polydimethylsiloxanes, polyphenylmethylsiloxanes, poly(diphenylsiloxanedimethylsiloxanes), poly(dimethylsiloxanemethylvinylsiloxanes), poly(dimethylsiloxanephenylmethylsiloxanes) or poly(diphenylsiloxanedimethylsiloxanemethylvinylsiloxanes).

These silicone gums may be terminated at a chain end with trimethylsilyl or dimethylhydroxysilyl groups.

The silicone resins that may be used in the compositions according to the present disclosure are crosslinked siloxane systems comprising units R₂SiO_{2/2}, RSiO_{3/2} or SiO_{4/2}, wherein R is chosen from hydrocarbon-based groups comprising from 1 to 6 carbon atoms and phenyl groups. In one embodiment of the present disclosure, R is chosen from lower (C₁-C₆) alkyl radicals and phenyl radicals.

The at least one nonionic compound may be present in an amount ranging from 0.1% to 20%, such as from 0.5% to 10%, for instance, from 0.6% to 5% by weight, relative to the total weight of the composition.

In another embodiment of the present disclosure, the cosmetic composition comprises at least one anionic fixing polymer of the type such as the copolymer of AMP acrylate/allyl methacrylate (e.g. Fixate G100 from the company Noveon) in an amount ranging from 0.6% to 5% by weight, relative to the total weight of the composition, at least one nonionic compound chosen from C₃-C₁₀ polyols, ethoxylated-fatty alcohols of HLB>5 and silicone copolyols in an amount ranging from 0.1% to 5% by weight, relative to the total weight of the composition, this composition being such that the weight ratio of the at least one anionic fixing polymer to the at least one nonionic compound is greater than 1.

The composition as disclosed herein may also comprise at least one adjuvant chosen from nonionic, anionic, cationic and amphoteric surfactants; nonionic, anionic, cationic and amphoteric additional polymers (other than the anionic fixing polymers described above); ceramides and pseudoceramides; vitamins and provitamins including panthenol; water-soluble and liposoluble, liquid and solid sunscreens; solid compounds such as pigments, nacreous agents and opacifiers; dyes; sequestering agents; plasticizers; solubilizers; acidifying agents; basifying agents; neutralizers; mineral and organic thickeners; antioxidants; hydroxy acids; solvents; penetrating agents; fragrances; buffers; dispersants; conditioning agents; opacifiers; and preserving agents.

The at least one adjuvant can be present in an amount for each adjuvant ranging from 0.01% to 20% by weight, relative to the weight of the composition.

Needless to say, a person skilled in the art will take care to select the optional additional compound(s) such that the beneficial properties intrinsically associated with the composition in accordance with the present disclosure are not, or are not substantially, adversely affected by the envisaged addition(s).

The appropriate medium of the compositions comprises water or of a mixture of water and of at least one organic solvent to dissolve the compounds that are not sufficiently water-soluble. Examples of organic solvents that may be used include hydroxylated or non-hydroxylated C₁-C₄ lower alcohols, such as ethanol and isopropanol; polyol ethers, for instance 2-butoxyethanol, propylene glycol, propylene glycol monomethyl ether, diethylene glycol monomethyl ether and monoethyl ether, aromatic alcohols, for instance benzyl alcohol or phenoxyethanol, and mixtures thereof.

The at least one solvent may be present in an amount ranging from 1% to 40% by weight, such as from 5% to 30% by weight, relative to the total weight of the dye composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The examples that follow serve to illustrate the invention without, however, being limiting in nature.

### EXAMPLES

The formulations of the following examples were prepared:

Formulation 1

| | |
|---|---|
| Steareth-21 | 1% |
| Alcohol | 40% |
| Fixate G100 (Noveon) | 1.5% AM |
| AMP | pH |
| Deionized water | qs 100 |
| Fragrance | qs |

Fixate G100 (Noveon) is a copolymer of AMP acrylates/allyl methacrylate.

Formulation 2

| | |
|---|---|
| Steareth-21 | 0.6% |
| Alcohol | 40% |
| Propylene glycol | 1.4% |
| Fixate G100 (Noveon) | 3.2% AM |
| AMP | pH |
| Deionized water | qs 100 |
| Fragrance | qs |

Formulation 3

| | |
|---|---|
| Dimethicone copolyol | 1% |
| (DC 1248) | |
| Alcohol | 40% |
| Fixate G100 (Noveon) | 3.15% AM |
| AMP | pH |
| Deionized water | qs 100 |
| Fragrance | qs |

Each of these formulations was applied to a panel of models and to locks of natural hair.

After an action time of about 30 seconds, a flat or round heating iron at 200° C. capable of shaping this hair was applied.

Two series of tests were performed:

The first series comprised smoothing each lock of hair treated with formulations 1, 2 and.3 using a flat iron (Techniliss Ioni Ceramic PNC 228 Velecta) by performing several passes in a continuous movement from the root to the end.

The second series comprised shaping the hair using curling irons.

Good hold of the hairstyle obtained was observed: the hair was soft and the locks are smooth and shiny, and in addition no deposit of unattractive whitish residues was observed.

1. A process for shaping keratin fibers, comprising:
applying to the keratin fibers a cosmetic composition comprising at least one anionic fixing polymer and at least one nonionic compound chosen from fatty alcohols, polyols and silicones chosen from polyorganosiloxanes comprising polyethyleneoxy and/or polypropyleneoxy groups, and
after an optional action time, placing the keratin fibers in contact with heat to shape the keratin fibers.

2. The process for shaping keratin fibers according to claim 1, wherein the action time ranges from 30 seconds to 10 minutes.

3. The process for shaping keratin fibers according to claim 1, wherein the heat is a temperature ranging from 50° C. to 250° C.

4. The process for shaping keratin fibers according to claim 3, wherein the heat is a temperature ranging from 100° C. to 230° C.

5. The process for shaping keratin fibers according to claim 1, wherein the at least one anionic polymer is chosen from anionic polymers comprising groups derived from carboxylic, sulfonic or phosphoric acid, and have a number-average molecular mass ranging from 500 to 5,000,000.

6. The process for shaping keratin fibers according to claim 5, such that the at least one anionic fixing polymer is chosen from homopolymers and copolymers of acrylic acid and methacrylic acid and the salts thereof; crotonic acid copolymers; copolymers of C₄-C₈ monounsaturated carboxylic acids and anhydrides; polyacrylamides comprising carboxylate groups; homopolymers and copolymers comprising sulfonic groups; anionic polyurethanes; and anionic grafted silicone polymers.

7. The process for shaping keratin fibers according to claim 1, wherein the at least one anionic polymer is chosen from copolymers comprising branched blocks comprising, as main monomers, at least one monomer chosen from C₁₋₂₀ alkyl acrylate, N-mono-(C₂₋₁₂ alkyl)(meth)acrylamide and N,N-di(C₂₋₁₂ alkyl)(meth)acrylamide, and at least one monomer chosen from acrylic acid and methacrylic acid.

8. The process for shaping keratin fibers according to claim 7, wherein the at least one anionic fixing polymer is a copolymer comprising branched blocks comprising, as monomers, n-butyl acrylate, acrylic acid, methacrylic acid and allyl methacrylate.

9. The process for shaping keratin fibers according to claim 1, wherein the at least one anionic fixing polymer is present in an amount ranging from 0.1% to 20% by weight, relative to the total weight of the composition.

10. The process for shaping keratin fibers according to claim 9, wherein the at least one anionic fixing polymer is present in an amount ranging from 0.6% to 5% by weight, relative to the total weight of the composition.

11. The process for shaping keratin fibers according to claim 1, wherein the at least one nonionic compound is a fatty alcohol comprising from 4 to 30 carbon atoms.

12. The process for shaping keratin fibers according to claim 11, wherein the at least one nonionic compound is a fatty alcohol comprising from 12 to 22 carbon atoms.

13. The process for shaping keratin fibers according to claim 11, such that the fatty alcohol is an ethoxylated fatty alcohol of HLB>5.

14. The process for shaping keratin fibers according to claim 1, wherein the at least one nonionic compound is a C₃-C₁₀ polyol comprising from 2 to 5 OH groups.

15. The process for shaping keratin fibers according to claim 14, wherein the polyol is chosen from glycerol, glycol, propylene glycol, dipropylene glycol, butylene glycol and butyl diglycol groups.

16. The process for shaping keratin fibers according to claim 1, wherein the at least one nonionic compound is chosen from linear, cyclic, branched and unbranched, volatile and non-volatile silicone.

17. The process for shaping keratin fibers according to claim 16, wherein the silicone is a polyorganosiloxane comprising polyethyleneoxy and/or polypropyleneoxy groups comprising C₆-C₂₄ alkyl groups.

18. The process for shaping keratin fibers according to claim 1, wherein the at least one nonionic compound is present in an amount ranging from 0.1% to 20% by weight, relative to the total weight of the composition.

19. The process for shaping keratin fibers according to claim 18, wherein the at least one nonionic compound is present in an amount ranging from 0.6% to 5% by weight, relative to the total weight of the composition.

20. The process for shaping keratin fibers according to claim 1, wherein the weight ratio of the at least one anionic fixing polymer to the at least one nonionic compound is greater than 1.

21. The process for shaping keratin fibers according to claim 1, wherein the cosmetic composition further comprises at least one adjuvant chosen from nonionic, anionic, cationic and amphoteric surfactants; nonionic, anionic, cationic and amphoteric additional polymers other than the at least one anionic polymer; ceramides and pseudoceramides; vitamins and provitamins; water-soluble and liposoluble, liquid and solid sunscreens; solid compounds; dyes; sequestering agents; plasticizers; solubilizers; acidifying agents; basifying agents; neutralizers; mineral and organic thickeners; antioxidants; hydroxy acids; solvents; penetrating agents; fragrances; buffers; dispersants; conditioning agents; opacifiers; and preserving agents.

22. A cosmetic composition for shaping keratin fibers comprising at least one anionic fixing polymer and at least one nonionic compound chosen from fatty alcohols, polyols and silicones,
wherein the at least one nonionic compound is present in an effective amount to reduce anionic polymer residues left on said keratin fibers during application of said composition to said keratin fibers in combination with heat.
